(19) United States
(12) Plant Patent Application Publication
(10) Pub. No.: US 2002/0073461 P1
(43) Pub. Date: Jun. 13, 2002

(54) VARIETY OF GERANIUM PLANT NAMED 'PACDALA'

(76) Inventor: Andrea Michalik, Dresden (DE)

Correspondence Address: Russell D. Orkin, WEBB ZIESENHEIM LOGSDON ORKIN & HANSON, P.C., 700 Koppers Building, 436 Seventh Avenue, Pittsburgh, PA, 15219-1818, US

(21) Appl. No.: 09/733,001

(22) Filed: Dec. 8, 2000

(51) Int. Cl. A01H 5/00

(52) U.S. Cl. PLT/328

(57) ABSTRACT

A new and distinct pelargonium plant with red colored flowers above medium green foliage. The plant is suitable for pot planting.

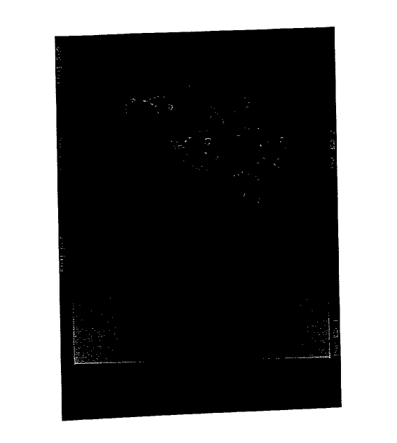

### BACKGROUND OF THE INVENTION

The present invention comprises a new and distinct cultivar of Pelargonium×hortorum known by the varietal name ‘Pacdala’. The new variety was discovered in 1997 in a selective breeding program in Dresden, Germany. The new variety is the result of cross breeding of a group of 25 dark-leafed and middle-green leafed seedlings having salmon colored flowers. The new variety was first asexually reproduced in 1998 by cuttings in Dresden, Germany. The new variety has been trial and field tested and has been found to retain its distinctive characteristics and remain true to type through successive propagations.

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Dresden, Germany. The color readings were taken outdoors in shadow during summer. Color references are primarily to the R.H.S. Colour Chart of The Royal Horticultural Society of London.

Plant:

Classification:

Botanical.—Pelargonium×hortorum.

Commercial.—Zonal geranium.

Form.—Erect.

Height from media surface to top of foliage.—35-40 cm.

Height from media surface to top of umbel.—50 cm.

Growth.—Good branching.

Strength.—Does not require artificial support.

Response time.—3.5 weeks.

Leaves:

Size.—8-10 cm long.

Shape.—Cordate.

Margin.—Partially toothed.

Texture.—Smooth; some pubescence.

Color.—Green Group 136A with faint zone.

Ribs and veins:

Color.—Darker leaf surface.

Petioles:

Color.—Medium green.

Length.—4-6 cm.

Stem/branches:

Color.—Medium green with dark red shine.

Internode length.—3-5 cm.

The bud:

Shape when just showing color:

Overall.—Spherical.

Individual bud.—Elliptical.

Color.—Yellow-Green Group 144A.

Size when just showing color:

Umbel.—8-10 cm across.

Individual bud.—1-1.5 cm long; 0.7-1.0 cm wide.

Inflorescence:

Blooming habit.—Semi-double.

Size of fully open bloom.—2.5-4 cm across.

Borne.—Flowers on umbel; umbel on pedicel; pedicel on peduncle.

Flowers:

Form.—Zygomorph.

Number of petals.—7-10 plus flakes.

Petals:

Size:

Upper petals.—1.5×2 cm.

Lower petals.—1.5×2 cm.

Color:

Upper surface.—Red Group 49A.

Lower surface.—Red Group 50D.

Texture and appearance.—Velvety.

Petaloids:

Quantity.—7.

Shape.—Flakes.

Color:

Upper surface.—Red Group 49A.

Lower surface.—Red Group 50D.

Pedicel:

Length.—2.5-3 cm.

Color.—Reddish-green.

Peduncle:

Length.—12-14 cm.

Color.—Reddish-green.

Disease/pest resistance.—No unusual susceptibility to diseases or pests has been noted to date.

Lasting Quality.—Long-lasting; 4 weeks per umbel.

Reproductive organs:

Stamens:

Anthers.—3 mm long.

Filaments:

Length.—0.5 cm maximum.

Color.—Pink.

Pollen.—Orange-red.

Pistils:

Number.—Five.

Length.—0.3-0.4 cm.

Stigma.—Pink.

Style.—0.8 cm long.

Ovaries:

Color.—Greenish.

Length.—0.4 cm.

Fruit.—Very rarely present.

### GENERAL CHARACTERISTICS

1. Dark-leafed.

2. Very insensitive against transport.

3. Good rooting.

4. Compact growth.

5. Stark contrast between leaf and flower color.

6. Very suitable for pots.

I claim:

1. A new and distinct variety of geranium plant substantially as shown and described.
